(19) United States
(12) Patent Application Publication
(10) Pub. No.: US 2025/0352257 A1
(43) Pub. Date: Nov. 20, 2025

(54) MAPPING AND ABLATING CATHETERS USING FLEXIBLE CIRCUITS

(71) Applicant: Boston Scientific Scimed, Inc., Maple Grove, MN (US)

(72) Inventor: Nathan Paul Hagstrom, Blaine, MN (US)

(21) Appl. No.: 19/208,163

(22) Filed: May 14, 2025

Related U.S. Application Data
(60) Provisional application No. 63/648,065, filed on May 15, 2024.

(51) Int. Cl. A61B 18/00

(52) U.S. Cl. CPC A61B 18/00; A61B 2018/00077; A61B 2018/00083; A61B 2018/00136; A61B 2018/0016; A61B 2018/00351; A61B 2018/00577; A61B 2018/00613; A61B 2018/00839

(57) ABSTRACT

A catheter for ablating cardiac tissue through irreversible electroporation, the catheter comprising an electrode assembly comprising a flexible circuit having a plurality of flex circuit branches extending proximally from a central hub portion, the flexible circuit further including an outwardly-facing ablation electrode including a plurality of ablation electrode branches, each of the ablation electrode branches extending proximally along a portion of a respective one of the flex circuit branches, each of the ablation electrode branches disposed on a dielectric flex circuit upper surface of the flexible circuit. A plurality of outwardly-facing spline sensing electrodes are located on each flex circuit branch; wherein one or more of the spline sensing electrodes on each flex circuit branch are disposed within a periphery of and spaced from the ablation electrode branch on the respective flex circuit branch by a gap, and wherein a dielectric coating is disposed within the gap and selectively covers portions of the ablation electrode branch and spline sensing electrode adjacent to the gap.

### CROSS REFERENCE TO RELATED APPLICATIONS

The present application claims priority to U.S. Provisional Patent Application No. 63/648,065, filed May 15, 2024, the entire contents of which are hereby incorporated by reference.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a catheter for use in ablating cardiac tissue through irreversible electroporation, the catheter comprising a tubular outer shaft and an electrode assembly. The tubular outer shaft has a proximal end and an opposite distal end. The electrode assembly extends distally from the distal end of the outer shaft, the electrode assembly defining a distally located central hub portion and a plurality of splines each including a distal end portion extending proximally from the central hub portion, a proximal end portion attached to and constrained by the outer shaft, and an intermediate portion between the proximal end portion and the distal end portion, the electrode assembly comprising a flexible circuit having a flex circuit hub and a plurality of flex circuit branches extending proximally from the flex circuit hub. The flexible circuit further includes an outwardly-facing ablation electrode and a plurality of outwardly-facing spline sensing electrodes located on each flex circuit branch. The ablation electrode includes a plurality of ablation electrode branches, each of the ablation electrode branches extending proximally along a portion of a respective one of the flex circuit branches and terminating in an ablation electrode proximal end, each of the ablation electrode branches disposed on a dielectric flex circuit upper surface of the flexible circuit. One or more of the spline sensing electrodes on each flex circuit branch are disposed within an outer periphery of and isolated from the ablation electrode branch on the respective flex circuit branch by a gap, and wherein a dielectric coating is disposed within the gap and selectively covers portions of the ablation electrode branch and spline sensing electrode adjacent to the gap.

In Example 2, the catheter of Example 1, wherein each ablation electrode branch includes one or more ablation electrode branch apertures formed therein, and wherein one of the spline sensing electrodes is disposed within a respective one of the ablation electrode branch apertures.

In Example 3, the catheter of Example 2, wherein each of the spline sensing electrodes has a major sensing surface and a sensing electrode sidewall, the major sensing surface and the sensing electrode sidewall defining a sensing electrode edge, and the sensing electrode sidewall and the dielectric flex circuit upper surface define a sensing electrode corner.

In Example 4, the catheter of Example 3, wherein each ablation electrode branch includes a major ablation electrode surface and an upstanding ablation electrode sidewall, the major ablation electrode surface and the ablation electrode sidewall defining an ablation electrode edge, and the ablation electrode sidewall and the dielectric flex circuit upper surface defining an ablation electrode corner, wherein each ablation electrode branch aperture is defined by a respective ablation electrode sidewall, and wherein each gap is defined by a spacing between a respective sensing electrode sidewall and an opposing ablation electrode sidewall.

In Example 5, the catheter of Example 4, wherein each ablation electrode corner, each ablation electrode edge, each sensing electrode corner, and each sensing electrode edge is covered by a portion of the dielectric coating.

In Example 6, the catheter of Example 5, wherein each ablation electrode sidewall and each sensing electrode sidewall are covered by the dielectric coating.

In Example 7, the catheter of any of Examples 1-6, wherein the dielectric coating is disposed on the flex circuit upper surface within each gap.

In Example 8, the catheter of any of Examples 1-7, wherein the dielectric coating is formed from parylene, polyvinylidene fluoride, or a polyimide resin.

In Example 9, the catheter of any of Examples 1-8, wherein the dielectric coating is deposited via one of sheet film, slot die, spray coating, dip coating, chemical vapor deposition, and atomic layer deposition.

In Example 10, the catheter of any of Examples 1-9, wherein the dielectric coating has a thickness of from about 3 micrometers to about 50 micrometers.

In Example 11, the catheter of Example 10, wherein the dielectric coating has a thickness of from about 3 micrometers to about 25 micrometers.

In Example 12, the catheter of Example 111, wherein the dielectric coating has a thickness of about 5 micrometers.

In Example 13, the catheter of any of Examples 1-12, wherein the electrode assembly further comprises a support member having a support member hub and a plurality of support member branches extending proximally from the support member hub, wherein each of the flex circuit branches is disposed over a respective one of the support member branches, and wherein the dielectric coating is further disposed over at least a portion of the support member.

In Example 14, the catheter of any of Examples 1-13, wherein each spline sensing electrode includes a biocompatible electrically-conductive plating on an electrically-conductive core, wherein a surface pattern is formed in the electrically-conductive plating to provide an increased effective sensing surface area.

In Example 15, the catheter of Example 14, wherein the surface pattern is formed via laser micro-etching.

In Example 16, a catheter for use in ablating cardiac by irreversible electroporation, the catheter comprising a tubular outer shaft having a proximal end and an opposite distal end, and an electrode assembly extending distally from the distal end of the outer shaft, the electrode assembly defining a distally located central hub portion and a plurality of splines each including a distal end portion extending proximally from the central hub portion, a proximal end portion attached to and constrained by the outer shaft. Each spline comprises a support member, and a flexible circuit disposed on the support member. The flexible circuit comprises a dielectric layer disposed on the support member and having an upper surface, an ablation electrode disposed on the upper surface of the dielectric layer, the ablation electrode including an aperture extending through the ablation electrode to the upper surface of the dielectric layer, a sensing electrode disposed on the upper surface of the dielectric layer within the aperture and spaced from the ablation electrode by a gap, wherein a dielectric coating is disposed within the gap and selectively covers portions of the ablation electrode branch and spline sensing electrode adjacent to the gap.

In Example 17, the catheter of Example 16, wherein the spline sensing electrode has a major sensing surface and a sensing electrode sidewall, the major sensing surface and the sensing electrode sidewall defining a sensing electrode edge, and the sensing electrode sidewall and the upper surface of the dielectric layer defining a sensing electrode corner.

In Example 18, the catheter of Example 17, wherein the ablation electrode includes a major ablation electrode surface and an upstanding ablation electrode sidewall, the major ablation electrode surface and the ablation electrode sidewall defining an ablation electrode edge, and the ablation electrode sidewall and the upper surface of the dielectric layer defining an ablation electrode corner, wherein the aperture is circumscribed by the ablation electrode sidewall, and wherein the gap is defined by a spacing between the sensing electrode sidewall and the ablation electrode sidewall.

In Example 19, the catheter of Example 18, wherein the ablation electrode corner, the ablation electrode edge, the sensing electrode corner, and the sensing electrode edge is covered by a portion of the dielectric coating.

In Example 20, the catheter of Example 19, wherein the ablation electrode sidewall and the sensing electrode sidewall are covered by the dielectric coating.

In Example 21, the catheter of Example 20, wherein the dielectric coating is disposed on the upper surface of the dielectric layer within each gap.

In Example 22, the catheter of Example 21, wherein the dielectric coating is formed from parylene, polyvinylidene fluoride, a polyimide resin.

In Example 23, the catheter of Example 22, wherein the dielectric coating is deposited via one of sheet film, slot die, spray coating, dip coating, chemical vapor deposition, and atomic layer deposition.

In Example 24, the catheter of Example 22, wherein the dielectric coating has a thickness of from about 3 micrometers to about 25 micrometers.

In Example 25, the catheter of Example 22, wherein the dielectric coating is further disposed over at least a portion of the support member.

In Example 26, the catheter of Example 16, wherein the sensing electrode includes a biocompatible electrically-conductive plating on an electrically-conductive core, wherein a surface pattern is formed in the electrically-conductive plating to provide an increased effective sensing surface area.

In Example 27, a catheter for use in ablating cardiac tissue through irreversible electroporation, the catheter comprising a tubular outer shaft having a proximal end and an opposite distal end, and an electrode assembly extending distally from the distal end of the outer shaft. The electrode assembly defines a distally located central hub portion and a plurality of splines each including a distal end portion extending proximally from the central hub portion, a proximal end portion attached to and constrained by the outer shaft, and an intermediate portion between the proximal end portion and the distal end portion, the electrode assembly comprising a flexible circuit having a flex circuit hub and a plurality of flex circuit branches extending proximally from the flex circuit hub. The flexible circuit further includes a dielectric layer having an upper surface, an outwardly-facing ablation electrode disposed on the upper surface of the dielectric layer and including a plurality of ablation electrode branches, each of the ablation electrode branches extending proximally along a portion of a respective one of the flex circuit branches, each of the ablation electrode branches including an aperture, and a plurality of outwardly-facing spline sensing electrodes located on each flex circuit branch, wherein one of the spline sensing electrodes on each flex circuit branch is disposed within the aperture of the respective ablation electrode branch and is spaced from the ablation electrode branch by a gap, and wherein a dielectric coating is disposed within the gap and selectively covers portions of the ablation electrode branch and the spline sensing electrode adjacent to the gap.

In Example 28, the catheter of Example 27, wherein each spline sensing electrode has a major sensing surface and a sensing electrode sidewall, the major sensing surface and the sensing electrode sidewall defining a sensing electrode edge, and the sensing electrode sidewall and the upper surface of the dielectric layer defining a sensing electrode corner.

In Example 29, the catheter of Example 28, wherein each ablation electrode includes a major ablation electrode surface and an upstanding ablation electrode sidewall, the major ablation electrode surface and the ablation electrode sidewall defining an ablation electrode edge, and the ablation electrode sidewall and the upper surface of the dielectric layer surface defining an ablation electrode corner, wherein each aperture is circumscribed by a respective ablation electrode sidewall, and wherein each gap is defined by a spacing between the respective sensing electrode sidewall and the respective ablation electrode sidewall.

In Example 30, the catheter of Example 29, wherein each ablation electrode corner, each ablation electrode edge, each sensing electrode corner, and each sensing electrode edge is covered by a portion of the dielectric coating.

In Example 31, the catheter of Example 30, wherein each ablation electrode sidewall and each sensing electrode sidewall are covered by the dielectric coating.

In Example 32, the catheter of Example 31, wherein the dielectric coating is disposed on the upper surface of the dielectric layer within each gap.

In Example 33, a catheter for use in ablating cardiac tissue through irreversible electroporation, the catheter comprising a tubular outer shaft having a proximal end and an opposite distal end, and an electrode assembly extending distally from the distal end of the outer shaft. The electrode assembly defines a distally located central hub portion and a plurality of splines each including a distal end portion extending proximally from the central hub portion, a proximal end portion attached to and constrained by the outer shaft, and an intermediate portion between the proximal end portion and the distal end portion, the electrode assembly comprising a flexible circuit having a flex circuit hub and a plurality of flex circuit branches extending proximally from the flex circuit hub. The flexible circuit further includes a dielectric layer having an upper surface, an outwardly-facing ablation electrode disposed on the upper surface of the dielectric layer and including a plurality of ablation electrode branches, each of the ablation electrode branches extending proximally along a portion of a respective one of the flex circuit branches, each of the ablation electrode branches including an aperture, and a plurality of outwardly-facing spline sensing electrodes located on the upper surface of the dielectric layer, wherein one of the spline sensing electrodes on each flex circuit branch is disposed within an outer periphery of and spaced from a surface of the ablation electrode branch on the respective flex circuit branch by a gap, and wherein a dielectric coating is disposed within the gap and selectively covers portions of the ablation electrode branch and the spline sensing electrode adjacent to the gap.

In Example 34 the catheter of Example 33, wherein the dielectric coating is disposed on the upper surface of the dielectric layer within each gap.

In Example 35, the catheter of Example 34, wherein the dielectric coating has a thickness of about 5 micrometers.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIGS. 2A and 2B are perspective and end view illustrations, respectively, of a distal portion of a splined catheter for use in the electrophysiology system of FIG. 1, in accordance with embodiments of the subject matter of the disclosure.

FIG. 2C is a partial plan view of an electrode assembly of the splined catheter shown in two-dimensions, in accordance with embodiments of the subject matter of the disclosure.

FIG. 2D is an enlarged plan view of a portion of a spline of the electrode assembly shown in FIGS. 2A-2C, in accordance with embodiments of the subject matter of the disclosure.

FIGS. 3A-3B are schematic cross-sectional views of a spline of the electrode assembly of FIG. 2B, in accordance with embodiments of the subject matter of the disclosure.

FIG. 4 is a schematic partial plan view of a sensing electrode, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

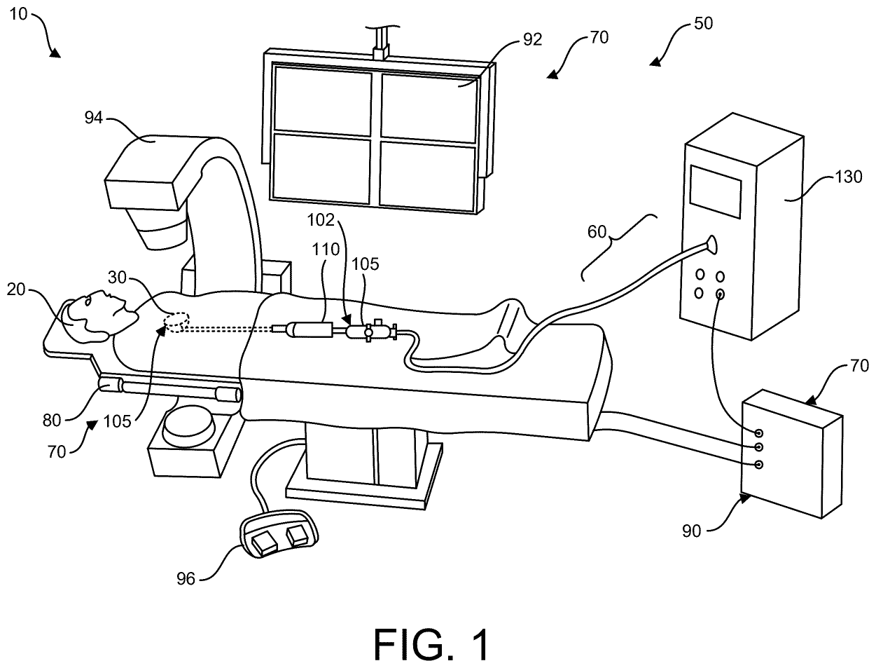

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation catheter system 60 includes an electroporation catheter 100 having a proximal portion 102 and a distal portion 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the electroporation catheter 100, in particular all or part of the distal portion 105 thereof, can be deployed to the specific target sites within the patient's heart 30.

In embodiments, the electroporation catheter system 60 is configured to deliver electric field energy to targeted tissue in the patient's heart 30 to create tissue apoptosis, rendering the tissue incapable of conducting electrical signals.

The electroporation console 130 is configured to control functional aspects of the electroporation catheter system 60. In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the electroporation catheter 100 in a monopolar or bipolar mode of operation, as will be described in further detail herein.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

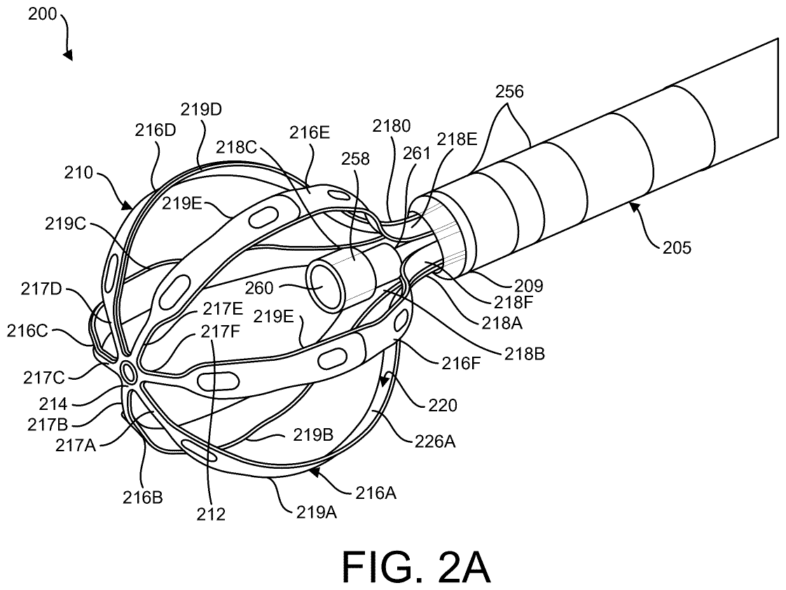

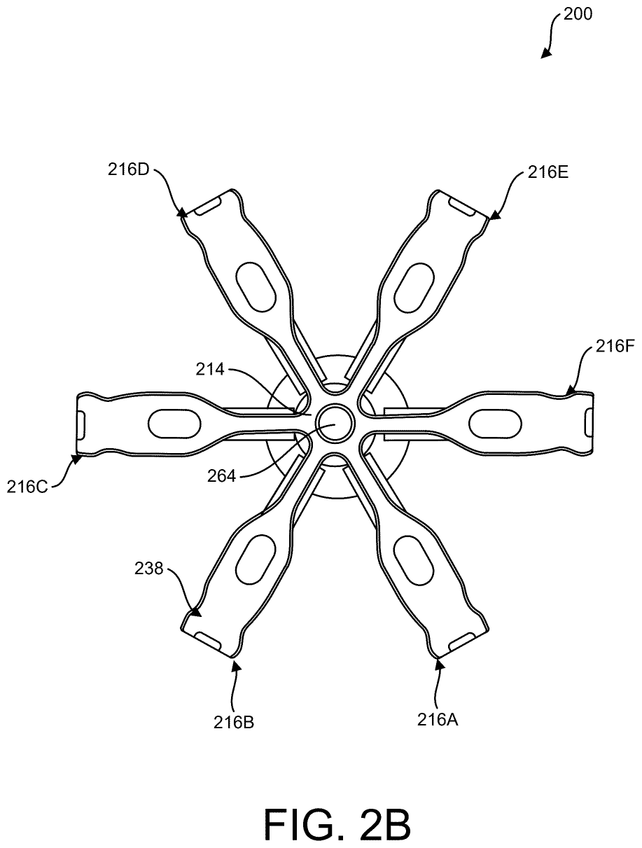

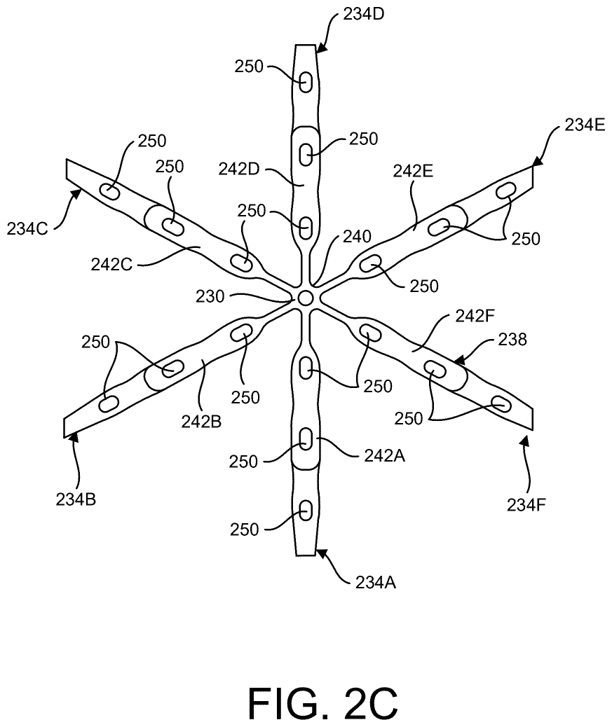

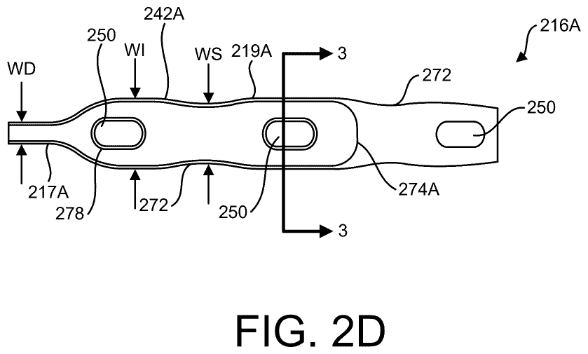

FIGS. 2A and 2B are partial perspective and end view illustrations, respectively, of an electroporation catheter 200 having a catheter distal portion 205 according to an embodiment of the present disclosure. The electroporation catheter 200 corresponds to the electroporation catheter 100 described with respect to FIG. 1. The electroporation catheter 200 has a tubular outer shaft 202 having a shaft distal end 209, and an electrode assembly 210 extending distally from the distal end 209 of the outer shaft 202. In embodiments, the electrode assembly 210 is configured to self-expand from a collapsed configuration when constrained within a delivery sheath to a pre-defined expanded configuration defining an inner space 212. As will be explained in greater detail herein, the electrode assembly 210 comprises an ablation electrode configured to receive pulsed electrical signals/waveforms from the electroporation console 130 (FIG. 1), thereby creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy.

Overall, the electrode assembly 210 and other electrode assembly embodiments described herein within the scope of the present disclosure, is primarily designed for the creation of relatively localized ablation lesions (i.e., focal lesions), as compared to relatively large diameter circumferential lesions created in pulmonary vein isolation procedures. However, the skilled artisan will appreciate that the teachings of the present disclosure can be readily adapted for a catheter capable of large diameter circumferential lesions. The designs of the various electrode assembly embodiments described herein can provide the clinician with a wide range of capabilities for monopolar and bipolar focal pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

FIG. 2C is a partial plan view of the electrode assembly 210 of the electroporation catheter 200 shown, shown in two-dimensions to illustrate the layout of the electrode assembly 210. Referring to FIGS. 2A-2C together, in the illustrated embodiment, the electrode assembly 210 as a whole has a distally-located central hub portion 214 and a plurality of splines 216A-216F extending proximally from the central hub portion 214. As further shown, each respective spline 216A-216F has a distal end portion 217A-217F, a proximal end portion 218A-218F, and an intermediate portion 219A-219F extending between the distal end portion 217A-217F and the proximal end portion 218A-218F. As shown, each of the proximal end portions 218A-218F is attached to and constrained by the distal end 209 of the outer shaft 202. As further shown, in the illustrated embodiment, the intermediate portion 219A-219F of each spline 216A-216F has a lateral width that is greater than the lateral width of each of the respective distal end portions 217A-217F. In embodiments, the particular geometry of the splines 216A-216F and the related components, e.g., ablation and mapping electrodes, is optimized to provide desired mechanical and therapeutic/diagnostic capabilities.

In the illustrated embodiment, the splines 216A-216F are composed of a support member 220 and a flexible circuit 222 secured to and disposed over an outer surface of the support member 220. The support member 220 functions, among other things, as a primary structural support of the electrode assembly 210, and thus primarily defines the mechanical characteristics of the electrode assembly 210. In embodiments, the support member 220 is formed from a superelastic material (metal or polymer) to provide desired mechanical/structural properties to the electrode assembly 210. In embodiments, the support member 220 is formed from a superelastic metal alloy, e.g., a nickel-titanium alloy.

The support member 220 includes a support member hub 224 and a plurality of support member branches (for ease of illustration, only support member branch 226A is labeled in FIG. 2A). In embodiments, the support member branches are integrally formed with and extend proximally from the support member hub 224. For example, the entire support member 200 may be cut from a single sheet of material using conventional manufacturing techniques. This unitary structure provides robust structural properties, for example, selective flexibility and enhanced fatigue characteristics, particularly in areas that are subject to relatively high stresses during manufacture and use of the electroporation catheter 200. Forming the support member 220 from a superelastic material such as a nickel-titanium alloy facilitates configuring the support member 220 to assume its desired unconstrained shape such as shown in FIG. 2A due to the shape memory properties of the material, while providing sufficient flexibility necessary to collapse the electrode assembly 210 within a delivery sheath. In embodiments, the support member branches can be selectively configured along their lengths to tune the mechanical characteristics of the electrode assembly 210.

The flexible circuit 222 includes a flex circuit hub 230 and a plurality of flex circuit branches 234A-234F. In embodiments, the flex circuit hub 230 is disposed over and secured to the support member hub 224. In embodiments, the flex circuit branches 234A-234F are integrally formed with the flex circuit hub 230, and each of the flex circuit branches 234A-234F is disposed over and secured to a respective one of the support member branches. The flexible circuit 222 comprises a layered construction including one or more dielectric substrate layers, and conductive traces formed thereon. Similar to the support member 220, the unitary construction of the flexible circuit 222 enhances its structural properties, for example, by minimizing joints or other discontinuities at regions subject to relatively high stresses during use.

As shown, the flexible circuit 222 includes an ablation electrode 238 that has an ablation electrode hub portion 240 and a plurality of ablation electrode branches 242A-242F. In the illustrated embodiment, the distal ablation electrode hub portion 240 is located on the flex circuit hub 230. Additionally, the ablation electrode branches 242A-242F are integrally formed with the ablation electrode hub portion 240. Each of the ablation electrode branches 242A-242F extends proximally along a portion of a respective one of the flex circuit branches 234A-234F.

As further shown, the flexible circuit 222 includes a plurality of spline sensing electrodes 250. In the illustrated embodiment, two of the spline sensing electrodes 250 are disposed within a periphery of each of the ablation electrode branches 242A-242F, and one of the spline sensing electrodes 250 is located proximal to each of the ablation electrode branches 242A-242F on a respective flex circuit branch 234A-234F. The illustrated configuration is exemplary only, and other embodiments of the catheter 200 may have alternative configurations. Thus, in various embodiments, one or more of the spline sensing electrodes 250 may be disposed within the periphery of one or more of the ablation electrode branches 242A-242F and electrically isolated therefrom, and one or more of the spline sensing electrodes 250 may be located proximal to the ablation electrode branches 242A-242F on the respective flex circuit branch 234A-234F. In still other embodiments, no spline sensing electrodes 250 may be located outside the peripheries of the ablation electrode branches 242A-242F.

In some embodiments, the structural functionality of the support member 220 can be provided by a suitably designed flexible circuit 222. As such, although the electrode assembly 210 is described in detail as including the support member 220 as a primary structural member, in other embodiments the support member 220 can be omitted in its entirety and the corresponding functionality can be provided by the flexible circuit 222.

In the particular illustrated embodiment, the electroporation catheter 200 includes a pair of shaft electrodes 256 located proximate the distal end 209 of the outer shaft 202, as well as a central post 258 extending distally from the distal end 209 of the outer shaft 202. As shown, the central post 258 extends partially into the inner space 212, and includes a post electrode 260. In embodiments, the central post 258 may house additional components. For example, in embodiments, a magnetic navigation sensor (not shown) may be partially or wholly disposed within the central post 258. However, in other embodiments such a sensor may be located elsewhere on the electroporation catheter 200 (e.g., within the outer shaft 202). In the illustrated embodiment, the electrode assembly 210 further includes a hub sensing electrode 264 centrally located on the flex circuit hub 230.

In embodiments, one or both of the shaft electrodes 256 can be configured to be paired with the ablation electrode 238 to form an anode/cathode ablation electrode pair for generation of an ablative electric field in a bipolar mode. In embodiments, the shaft electrodes 256 may have additional functions, e.g., and without limitation, as additional sensing electrodes for sensing cardiac electrical signals, and for use as localization sensors for impedance tracking of the electrode assembly 210.

The post electrode 260 can provide a number of functional advantages. In one example, the post electrode 260 can operate as a reference for unipolar electrograms, in lieu of reliance on surface ECG patch electrodes as are otherwise known in the art. The location of the post electrode 260 for this purpose positions the reference electrode much closer to the tissue being sensed than is possible with the conventional surface ECG approach, which may advantageously minimize far field noise and provide much sharper unipolar electrograms than what are possible using surface ECG electrodes. The post electrode 260 may also be operable to sense and measure other electrical parameters, e.g., voltages between it and the ablation electrodes or other sensing electrodes on the electrode assembly 210, thereby providing data usable for, in some examples, determining the shape of the electrode assembly during use (including when deformed by forces applied by cardiac walls), and displaying shape information via the EAM system 70 (FIG. 1).

In embodiments, the hub sensing electrode 264 allows tissue surface mapping to be conducted in a “forward” manner, eliminating the need to manipulate the electrode assembly 210 to place the spline sensing electrodes 250 against or proximate the tissue to be mapped. The inclusion of the hub sensing electrode 264 further enhances bipolar sensing capabilities by providing for, in the illustrated embodiment, six additional bi-poles when paired with any of the distal-most spline sensing electrodes 250.

FIG. 2D is an enlarged plan view of a portion of the spline 216A, the ablation electrode branch 242A, and the flex circuit branch 234A, according to embodiments of the present disclosure. The structural features illustrated in FIG. 2D are representative the splines 216A-216F, the ablation electrode branches 242A-242F and the flex circuit branches 234A-234F.

As shown, the distal end portion 217A of the spline 216A has a maximum width WD, and the intermediate portion 219A of the spline 216A has a maximum width W1 that is greater than the maximum width WD of the distal end portion. In the particular embodiment shown, the intermediate portion 219A further includes one or more scalloped regions 272 wherein the opposing outer edges of the spline 216A have a concave shape. In embodiments, the scalloped regions 272 are selectively located along the length of the spline 216A and each have a scalloped region minimum width WS that is less than the maximum width W1 of the intermediate portion 219A. When present, the scalloped regions 272 affect the mechanical properties (e.g., bending flexibility) of the spline 216A, to, for example, facilitate deformation of the spline 216A when it is in contact with target tissue, as well as facilitating collapse of the electrode assembly 210 when it is retracted into a delivery sheath. However, in some embodiments, the scalloped regions 272 are omitted, and the spline 216A has a generally linear shape along the intermediate portion 219A. In the illustrated embodiment, at least one of the scalloped regions 272 is located in the region of the spline 216A on which a portion of the ablation electrode branch 242A is disposed, and between the spline sensing electrodes 250 located thereon.

As shown, the ablation electrode branch 242A has a proximal end 274A. In the illustrated embodiment, the proximal end 274A is contoured and shaped to enhance electric field generation and clinical efficacy when the catheter 200 is configured to operate in bi-polar energy delivery mode, with the ablation electrode 238 and one or both of the shaft electrodes 256 paired as a bi-polar electrode pair. In other embodiments, however, the proximal end 274A can take on different shapes, e.g., semi-circular. The location of the proximal end 274A (which as will be appreciated, defines the length of the ablation electrode branch 242A and consequently defines, in part, the overall surface area of the ablation electrode 238) can be varied from embodiment to embodiment depending on the particular clinical needs required of the catheter 200.

As further shown, the ablation electrode branch 242A includes a plurality of ablation electrode branch apertures 278, and one of the spline sensing electrodes 250 is disposed within each of the ablation electrode branch apertures 278.

The application of the flexible circuit 222 for use with the ablation electrode 238 and spline sensing electrodes 250 provides for several advantages but can also introduce challenges in maintaining ablation and sensing performance. For instance, portions of the flexible circuit can be susceptible to damage from ablation, and the high voltages required for electroporation may generate electrical noise that can impact sensing effectiveness. Additionally, the construction of the flex circuit and corresponding electrodes themselves can lend to noise issues. Electrodes disposed on dielectric flexible circuit substrates or layers are thin, but still include three-dimensional geometries such as heights in a direction perpendicular to the surfaces of the dielectric layers, such as perpendicular to the width and length dimensions described above. For example, a sidewall height to the major surface of a flexible circuit ablation or sensing electrode is approximately 12-25 micrometers above the uppermost dielectric layer of the flexible circuit 222.

When flexible circuit electrodes are used to delivery PFA energy as ablation electrodes, the geometry can create localized heating at areas proximate the corner of the sidewall and major surface and the proximate the edge of the sidewall and dielectric substrate. The localized heating can generate bubbles in the blood and tissue. The localized heating can also cause electrode erosion due to “corner weakness,” which is a phenomenon that occurs when relatively thick layers of electrodeposited metal results in thin depiction at electrode corners. In addition, the flexible circuit 222 having a sensing electrode 250 in the apertures 278 spaced from the ablation electrodes 238 can form a passive fluid channel where fluid heats and is not dissipated, which caused further heating and bubble formation.

When flexible circuit electrodes are used as sensing electrodes 250, the relatively small surface areas and microscale sidewalls present challenges with respect to sensing electrical noise. For instance, relatively small surface areas present a challenge as they create high impedances. Microscale sidewalls present issues with transient wetting of the electrode surface. In addition, the flexible circuit 222 having a sense electrode 250 in the aperture 278 spaced from the ablation electrode 238 can create a fluid channel where the blood/gas mixture has a transient nature, making electrode wetting less consistent.

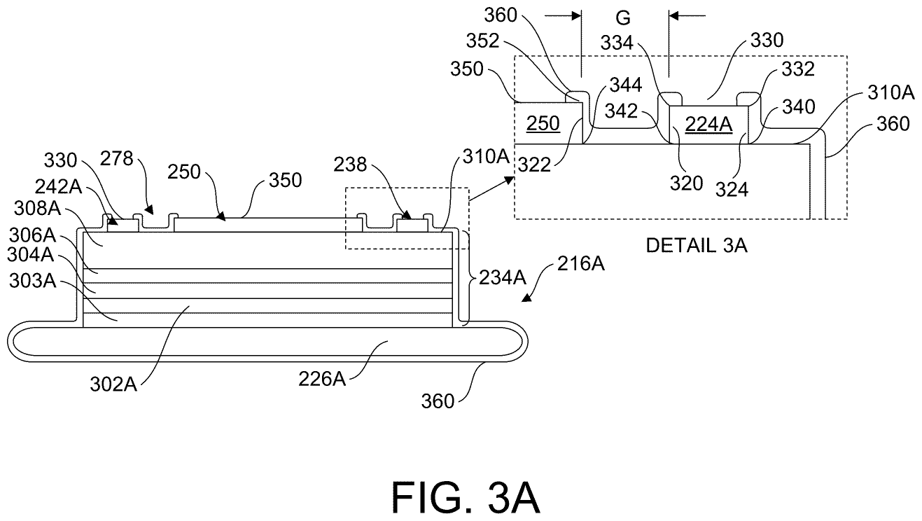

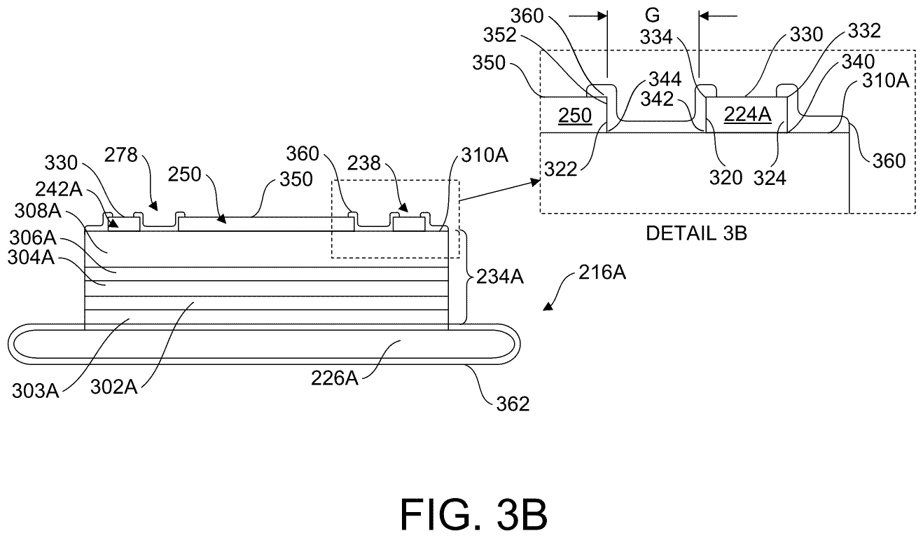

FIGS. 3A-3B are schematic cross-sectional views of the spline 216A taken along the line 3-3 in FIG. 2D, illustrating an exemplary configurations of the flex circuit branch 234A to mitigate bubble formation when pulsed field ablation energy is delivered. With reference to FIGS. 3A and 3B collectively, as well as Detail 3A and Detail 3B, the spline 216A includes the support member branch 226A and the flex circuit branch 234A is disposed thereon. As further shown, the flex circuit branch 234A comprises a layered structure that, except as specifically distinguished herein, may be typical of flexible circuits for use in medical device electrode assemblies. The layered structure and layers are presented for illustration and are not to scale. Additionally, the configuration of the spline 216A is presented for illustration of the electrode assembly 210, and is representative of the other splines thereof. It is emphasized, however, that the particular configuration of the flex circuit illustrated in FIGS. 3A-3B, for example, the particular stacking arrangement illustrated therein, is exemplary only, and in various embodiments, other flex circuit designs may be employed.

In the particular embodiments illustrated in FIGS. 3A and 3B, the flex circuit branch 234A includes a dielectric base layer 302A disposed over the support member branch 226A. In the illustrated embodiment, the dielectric base layer 302A is secured to the support member branch 226A by an adhesive layer 303A. In embodiments, the dielectric base layer 302A may be formed from a polyimide (Pl) dielectric material, and the adhesive layer 303A is an acrylic adhesive 303A. An optional inner flexible adhesive layer 304A is deposited over the base layer 302A. A conductive trace layer 306A is disposed over the adhesive layer 304A (when present). A dielectric upper layer 308A, is positioned over the conductive trace layer 306A, and includes an upper surface 310A. The dielectric materials chosen for the layers 302A and 308A can be any conventional materials suitable for use in flexible circuits for medical devices and can include polyamides. It is emphasized that the present disclosure is not limited to the particular flex circuit stacking arrangement illustrated in FIGS. 3A and 3B, and that the skilled artisan will readily understand alternative arrangements that may be utilized.

In embodiments, the thicknesses of the various components of the spline 216A may be tailored for the particular structural and clinical needs for the electroporation catheter. In one illustrative example, the support member branch 226A may have a thickness of about 68-70 micrometers, including in one embodiment a thickness of about 68.8 micrometers. In embodiments, the dielectric layer 302A and the adhesive layers 303A, 304A may each have a thickness of about 12.7 micrometers, and the copper trace layer 306A may have a thickness of about 9 micrometers. In an exemplary embodiment, the upper dielectric layer 308A may be about 25.4 micrometers.

As further shown, the ablation electrode branch 242A and the spline sensing electrode 250 are disposed on the upper layer 308A. In some embodiments, the electrodes 238 and 250 include a biocompatible electrically-conductive plating on an electrically-conductive core. For example, the electrodes 238 and 250 are constructed from copper having a coating of a suitable biocompatible metal such as gold. For example, the copper electrode core can be approximately 9 micrometers thick and surrounded by a 2 micrometer gold plating. In embodiments, the outer surfaces of the electrodes 238 and 250 may be treated to provide the electrical properties desired for the particular clinical application, as explained in greater detail elsewhere herein.

As illustrated in FIGS. 3A and 3B, and in particular, Detail 3A and Detail 3B, respectively, the ablation electrode aperture 278 is bounded by an inner peripheral sidewall 320 of the proximal ablation electrode branch 242A, and an outer peripheral sidewall 322 of the spline sensing electrode 250 is spaced from the inner peripheral sidewall 320 of the ablation electrode branch 242A by a gap G. In embodiments, the gap G can range from about 0.050 to 0.50 millimeters. In one embodiment, the gap G is about 0.50 millimeters. Further, the ablation electrode branch 242A includes an outside sidewall 324 around the outer periphery of the ablation electrode branch 242A.

The ablation electrode branch 242A includes an outwardly facing major ablation surface 330 disposed between the upstanding sidewalls 320, 324. The outside sidewall 324 and major ablation surface 330 form, or define, an outer ablation electrode edge 332. The inner peripheral sidewall 320 and major ablation surface 330 form, or define, an inner ablation electrode edge 334. Also, the outside sidewall 324 and upper surface 310A of dielectric upper layer 308A form an outside ablation electrode corner 340. The inner peripheral sidewall 320 and upper surface 310A form or define an inner ablation electrode corner 342.

The sensing electrode 250 includes an outwardly facing major sensing surface 350 disposed between the upstanding inner peripheral sidewall 322. The inner peripheral sidewall 322 and major sensing surface 350 form, or define, a sensing electrode edge 352. The inner peripheral sidewall 322 and upper surface 310 form or define sensing electrode corner 344.

In embodiments, a thin dielectric coating 360 is selectively disposed over the sidewalls 320, 322, 324, edges 332, 334, 352, and corners 340, 342, 344, and within the gap G. In the embodiment, the thickness of the thin film dielectric coating 360 is selected to only partially fill the height of the gap G, so as to form a depressed racetrack with respect to the major ablation surface 330 and major sensing surface 350. In embodiments, the coating 360 may have a thickness of from about 3 micrometers to about 50 micrometers. In some embodiments, the dielectric coating has a thickness of from about 3 micrometers to about 25 micrometers. In one embodiment, the dielectric coating is a 5 micrometer thick coating, such that over 50% of the electrode height from the upper surface 310A to the major surfaces 330, 350 extend above the dielectric coating deposited on the surface 310A. In the illustrated example, the coating 360 is deposited over a small portion of the major ablation surface 330 and the major sensing surface 350 near the edges 332, 334, 352 and on the upper surface 310A in the gap G.

The dielectric coating 360 operates to reduce localized heating leading to bubble production during ablation and reduces sensing noise during mapping procedures of the electrode assembly 210. Dielectrically insulating the electrode corners 340, 342, 344, edges 332, 334, 352 and the gap G between ablation electrodes and sensing electrodes 250 reduces the amount localized electrode heating while reducing an opportunity for gases to collect in the gap G and other cavities during delivery of ablation energy. Partially filling the gap G with dielectric materials results in the sensing electrodes 250, whether used independently or concurrently with the application of ablation energy, to have a more consistent surface wetting with respect to time and motion, and reducing the opportunity for gases to collect in the gap G. In embodiments, the dielectric strength of the coating 360 is selected to overcome, or be greater than, the voltage applied to the electrodes during ablation.

The material for the dielectric coating 360 may be selected from a range of materials. In various embodiments, exemplary materials for the dielectric coating 360 may include a range of polyimides, parylene, polyvinylidene fluoride, acrylic adhesives, or other materials having comparable dielectric properties. The dielectric coating 360 can be deposited in a number of ways including via sheet film, slot die, spray coating, dip coating, chemical vapor deposition (CVD), and atomic layer deposition (ALD). In one example, the coating 360 can be processed in a sufficiently accurate manner to create borders starting at the corners 340, 342, 344 on the electrodes 250, 238, and extend on the major surfaces 330, 350 about 20-75 micrometers via photo-imaging, laser processing, and filling gaps G.

In the embodiment of FIG. 3A, the dielectric coating is disposed over substantially the entire surface of the spline 216A, with only the major ablation surface 330 and the major sensing surface 350 being uncoated. In the embodiment of FIG. 3B, the coating 360 can be more selectively applied and/or removed after application such that only the edges 332, 334, 352, the corners 340, 342, 344 and the gap G are covered by the coating 360 and the sidewall of the flex circuit branch 234 is uncoated. As further shown in FIG. 3B, in embodiments, the support member branch 226A may optionally include a separate coating of dielectric material.

It is emphasized that while FIGS. 3A and 3B and the corresponding paragraphs illustrate and describe the inclusion of the dielectric coating 360 to the spline sensing electrodes 250 and adjacent ablation electrode branches 242, the same construction is equally useful in respect of the hub sense electrode 264 and adjacent portions of the ablation electrode 238.

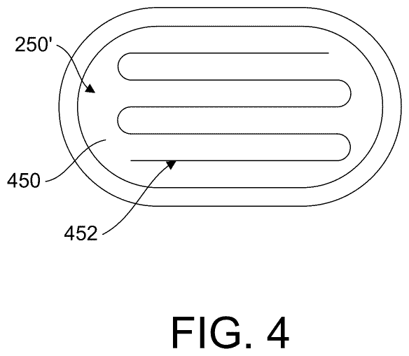

FIG. 4 schematically illustrates an embodiment of a sensing electrode 250′ having features to mitigate noise production during sensing, which can be utilized in any of the embodiments of the disclosure. In some embodiments, the electrode 250′ is constructed of a biocompatible electrically-conductive plating on an electrically-conductive core. For example, the electrode 250′ may be constructed from a copper core having a coating of a suitable biocompatible metal such as gold. For example, the copper electrode core can be approximately 9 micrometers thick and surrounded by a 2 micrometer gold plating. In the illustrated embodiment, the sensing electrode 250′ includes an outwardly facing major sensing surface 450 having a surface feature designed to increase the effective surface area of the electrode 250′, similar to fractal coatings applied to stimulation lead electrodes. In FIG. 4, the surface feature is depicted schematically as a generally serpentine pattern of lateral grooves 452 formed into the biocompatible plating, or biocompatible core if applicable, of the electrode 250′, although the skilled artisan will readily recognize that the illustrated pattern is exemplary only, and that the surface feature may take on other forms, including randomly distributed features.

The grooves 452 in major sensing surface 450 increases the surface roughness relative to an untreated surface, which consequently increases surface area of the sensing electrode 250′ while maintaining a homogenous sensing electrode construction that emulates a fractal coating. In one example, the surface feature, e.g., the grooves 452, may be formed in the major sensing surface 450 via a laser micro-etching process. The grooves 452 serve to lower impedance on the electrode 250′ as compared to a sensing electrode with a smooth major surface, while increasing consistency of electrode impedance across a variety of frequencies.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A catheter for use in ablating cardiac by irreversible electroporation, the catheter comprising:
a tubular outer shaft having a proximal end and an opposite distal end; and
an electrode assembly extending distally from the distal end of the outer shaft, the electrode assembly defining a distally located central hub portion and a plurality of splines each including a distal end portion extending proximally from the central hub portion, a proximal end portion attached to and constrained by the outer shaft, each spline comprising:
a support member;
a flexible circuit disposed on the support member, the flexible circuit comprising:
a dielectric layer disposed on the support member and having an upper surface;
an ablation electrode disposed on the upper surface of the dielectric layer, the ablation electrode including an aperture extending through the ablation electrode to the upper surface of the dielectric layer; and
a sensing electrode disposed on the upper surface of the dielectric layer within the aperture and spaced from the ablation electrode by a gap, wherein a dielectric coating is disposed within the gap and selectively covers portions of the ablation electrode branch and spline sensing electrode adjacent to the gap.

2. The catheter of claim 1, wherein the spline sensing electrode has a major sensing surface and a sensing electrode sidewall, the major sensing surface and the sensing electrode sidewall defining a sensing electrode edge, and the sensing electrode sidewall and the upper surface of the dielectric layer defining a sensing electrode corner.

3. The catheter of claim 2, wherein the ablation electrode includes a major ablation electrode surface and an upstanding ablation electrode sidewall, the major ablation electrode surface and the ablation electrode sidewall defining an ablation electrode edge, and the ablation electrode sidewall and the upper surface of the dielectric layer defining an ablation electrode corner, wherein the aperture is circumscribed by the ablation electrode sidewall, and wherein the gap is defined by a spacing between the sensing electrode sidewall and the ablation electrode sidewall.

4. The catheter of claim 3, wherein the ablation electrode corner, the ablation electrode edge, the sensing electrode corner, and the sensing electrode edge is covered by a portion of the dielectric coating.

5. The catheter of claim 4, wherein the ablation electrode sidewall and the sensing electrode sidewall are covered by the dielectric coating.

6. The catheter of claim 5, wherein the dielectric coating is disposed on the upper surface of the dielectric layer within each gap.

7. The catheter of claim 6, wherein the dielectric coating is formed from parylene, polyvinylidene fluoride, a polyimide resin.

8. The catheter of claim 7, wherein the dielectric coating is deposited via one of sheet film, slot die, spray coating, dip coating, chemical vapor deposition, and atomic layer deposition.

9. The catheter of claim 7, wherein the dielectric coating has a thickness of from about 3 micrometers to about 25 micrometers.

10. The catheter of claim 7, wherein the dielectric coating is further disposed over at least a portion of the support member.

11. The catheter of claim 1, wherein the sensing electrode includes a biocompatible electrically-conductive plating on an electrically-conductive core, wherein a surface pattern is formed in the electrically-conductive plating to provide an increased effective sensing surface area.

12. A catheter for use in ablating cardiac tissue through irreversible electroporation, the catheter comprising:
a tubular outer shaft having a proximal end and an opposite distal end; and
an electrode assembly extending distally from the distal end of the outer shaft, the electrode assembly defining a distally located central hub portion and a plurality of splines each including a distal end portion extending proximally from the central hub portion, a proximal end portion attached to and constrained by the outer shaft, and an intermediate portion between the proximal end portion and the distal end portion, the electrode assembly comprising a flexible circuit having a flex circuit hub and a plurality of flex circuit branches extending proximally from the flex circuit hub, the flexible circuit further including:
a dielectric layer having an upper surface;
an outwardly-facing ablation electrode disposed on the upper surface of the dielectric layer and including a plurality of ablation electrode branches, each of the ablation electrode branches extending proximally along a portion of a respective one of the flex circuit branches, each of the ablation electrode branches including an aperture; and
a plurality of outwardly-facing spline sensing electrodes located on each flex circuit branch, wherein one of the spline sensing electrodes on each flex circuit branch is disposed within the aperture of the respective ablation electrode branch and is spaced from the ablation electrode branch by a gap, and wherein a dielectric coating is disposed within the gap and selectively covers portions of the ablation electrode branch and the spline sensing electrode adjacent to the gap.

13. The catheter of claim 12, wherein each spline sensing electrode has a major sensing surface and a sensing electrode sidewall, the major sensing surface and the sensing electrode sidewall defining a sensing electrode edge, and the sensing electrode sidewall and the upper surface of the dielectric layer defining a sensing electrode corner.

14. The catheter of claim 13, wherein each ablation electrode includes a major ablation electrode surface and an upstanding ablation electrode sidewall, the major ablation electrode surface and the ablation electrode sidewall defining an ablation electrode edge, and the ablation electrode sidewall and the upper surface of the dielectric layer surface defining an ablation electrode corner, wherein each aperture is circumscribed by a respective ablation electrode sidewall, and wherein each gap is defined by a spacing between the respective sensing electrode sidewall and the respective ablation electrode sidewall.

15. The catheter of claim 14, wherein each ablation electrode corner, each ablation electrode edge, each sensing electrode corner, and each sensing electrode edge is covered by a portion of the dielectric coating.

16. The catheter of claim 15, wherein each ablation electrode sidewall and each sensing electrode sidewall are covered by the dielectric coating.

17. The catheter of claim 16, wherein the dielectric coating is disposed on the upper surface of the dielectric layer within each gap.

18. A catheter for use in ablating cardiac tissue through irreversible electroporation, the catheter comprising:
a tubular outer shaft having a proximal end and an opposite distal end; and
an electrode assembly extending distally from the distal end of the outer shaft, the electrode assembly defining a distally located central hub portion and a plurality of splines each including a distal end portion extending proximally from the central hub portion, a proximal end portion attached to and constrained by the outer shaft, and an intermediate portion between the proximal end portion and the distal end portion, the electrode assembly comprising a flexible circuit having a flex circuit hub and a plurality of flex circuit branches extending proximally from the flex circuit hub, the flexible circuit further including:
a dielectric layer having an upper surface;
an outwardly-facing ablation electrode disposed on the upper surface of the dielectric layer and including a plurality of ablation electrode branches, each of the ablation electrode branches extending proximally along a portion of a respective one of the flex circuit branches, each of the ablation electrode branches including an aperture; and
a plurality of outwardly-facing spline sensing electrodes located on the upper surface of the dielectric layer, wherein one of the spline sensing electrodes on each flex circuit branch is disposed within an outer periphery of and spaced from a surface of the ablation electrode branch on the respective flex circuit branch by a gap, and wherein a dielectric coating is disposed within the gap and selectively covers portions of the ablation electrode branch and the spline sensing electrode adjacent to the gap.

19. The catheter of claim 18, wherein the dielectric coating is disposed on the upper surface of the dielectric layer within each gap.

20. The catheter of claim 19, wherein the dielectric coating has a thickness of about 5 micrometers.
